(19) United States
(12) Patent Application Publication
(10) Pub. No.: US 2006/0276782 A1
(43) Pub. Date: Dec. 7, 2006

(54) Nerve stimulator for use as a surgical guide

(76) Inventor: Tewodros Gedebou, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 11/145,851

(22) Filed: Jun. 6, 2005

(51) Int. Cl. A61B 18/18

(52) U.S. Cl. 606/45

(57) ABSTRACT

Device and method for stimulating nerves while performing surgery to release a ligament are disclosed. According to a preferred embodiment, the device comprises an elongate first blade guide arm coupled to a second blade guide arm of substantially similar shape. At least one of the blade guide arms supports a nerve stimulation electrode at its distal end. The distal ends of the first and second blade guide arms are positioned about a ligament such that the device encloses a ligament, or a portion thereof. An electrical current is sent from an electrical source to the nerve stimulation electrode such that if a nerve is entrapped within the device, the nerve will cause a connected muscle to respond. A surgeon may then reposition the device to avoid severing the nerve. A blade is engaged to at least one of the blade guide arms, and moves parallel to a longitudinal axis of the blade guide arm to cut the ligament.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

There are currently about eight million patients with carpal tunnel syndrome and close to 250,000 operations are performed annually in the United States to relieve the painful condition. These numbers are only expected to increase given the substantial increase in the use of computers and to the poor ergonomics of the hand in relation to the computer.

The transverse carpal ligament, which forms the carpal tunnel, is located near the base of the palm. Tendons stretching from the forearm to the hand have to pass through the carpal tunnel to get into the hand. The median nerve also passes through this narrow canal and is the only nerve to traverse the tunnel. The median nerve provides sensation of feeling to the thumb, index and middle fingers and half of the ring finger. It also controls the working of some of the thumb muscles, for examples, musles that permit a person to pinch.

Carpal tunnel syndrome is often the result of a combination of factors that increase pressure on the median nerve in the carpal tunnel. The most common cause of pressure on the median nerve is inflammation of the tendons due to repetitive use of the hand and wrist. Repetitive use causes tendons to swell and the swelling narrows the tunnel which causes the median nerve to be compressed. Workers in industries such as the meat-packing industry, other industries that utilize assembly line production, or workers that consistently use vibrating hand tools are prone to show symptoms of carpal tunnel syndrome. Even industries such as dentistry are reporting a greater number of instances of carpal tunnel syndrome. Fluid retention during pregnancy is also a common cause of carpal tunnel syndrome. The result in any such case may be pain, weakness, or numbness in the hand and wrist.

Initial treatment of carpal tunnel syndrome generally involves immobilizing or resting the affected hand and wrist for a couple weeks and avoiding activities that may cause further swelling in the tunnel. A patient may also choose to immobilize the wrist in a splint to relieve pressure on the median. Stretching and strengthening exercises can also be helpful in treating people, but only after the patient's symptoms have waned. For more advanced cases, carpal tunnel release surgery is an option and is one of the most common surgical procedures in the United States.

The traditional surgical procedure used to treat carpal tunnel syndrome is open release surgery and is performed on an out-patient basis. This surgery consists of making a one and one half to two and one half inch incision from the base of the palm and extending away from the wrist. The incision exposes the transverse carpal ligament that forms the carpal tunnel. The surgeon then cuts the transverse carpal ligament which immediately reduces the pressure on the median nerve.

Even though the surgery is performed with only a local anesthetic and is on an out patient basis, the procedure carries extreme risks. Because it sits adjacent to the transverse carpal ligament, the median nerve is in danger of being severed during surgery. The median nerve, if inadvertently severed during surgery, can lead to significant disability of the patient including the restriction of mobility in the ring, middle and index fingers, as well as the thumb. The use of endoscopic procedures is not as widespread as predicted primarily due to the fear of nerve injury.

The current invention greatly improves upon the prior art by alleviating the fear of nerve damage during surgery. The current invention uses a ligament hugging surgical guide lined with a metal or other conductive material to transmit a low energy electrical impulse to signal if the median nerve is entrapped. If the nerve is entrapped, the patient will exhibit a motor response, such as a thumb twitch or jerk of the finger, and the surgeon will know not to proceed. The surgeon can then reposition the surgical guide until no motor response is detected and hence the nerve is not entrapped within the cutting device. Once the cutting device is aligned properly, the ligament can be cut either by a scissor or retractable blade technique.

The invention may also be used in different types of surgeries, such as deep plane face lifting, where there is significant risk of damaging a motor nerve. The current invention allows surgeons to accurately release ligaments that are adjacent to or positioned near nerves whose damage would severely inhibit the patient.

### BRIEF SUMMARY OF THE INVENTION

The present invention specifically addresses and alleviates the above-identified deficiencies in the art. In this regard, the present invention is directed to a device and method for stimulating nerves while performing surgery to release a ligament in order to prevent the inadvertent severing of nerves. According to a preferred embodiment, the device comprises an elongate first blade guide arm coupled to a second blade guide arm of substantially similar shape. At least one of the blade guide arms supports a nerve stimulation electrode at its distal end. The distal ends of the first and second blade guide arms are positioned about a ligament such that the device encloses a ligament, or a portion thereof. An electrical current is sent from an electrical source to the nerve stimulation electrode. If the electrode comes into contact with a nerve, such that the nerve is entrapped within the device, the nerve will cause a connected muscle to respond. A surgeon may then reposition the device to avoid severing the nerve. A blade is engaged to at least one of the blade guide arms, and moves parallel to a longitudinal axis of the blade guide arm to cut the ligament.

It is therefore an object of the present invention to provide a surgical guide device that stimulates nerves so that a doctor may accurately cut a ligament, or other connective tissue, without severing adjacent nerves.

Another object of the present invention to provide a surgical guide device that can be adapted to sever ligaments that are located in a variety of target sites about the body.

A still further object of the present invention is to provide a surgical guide device that stimulates nerves so that a doctor may accurately cut a ligament wherein the device is of simple construction, easy to use, and can be manufactured at relatively low cost.

### BRIEF DESCRIPTION OF THE DRAWINGS

These as well as other features of the present invention will become more apparent upon reference to the drawings.

FIG. 1 is a side view of a surgical guide device for cutting a ligament constructed in accordance with a preferred embodiment of the present invention, the device being operatively connected to an electrical source and having a retractable blade in accordance with a preferred embodiment of the invention.

FIG. 2 is a side view of the device of FIG. 1, shown with the blade of FIG. 1 fully projected. FIG. 2a is an enlarged view of the blade from FIG. 2.

FIG. 3 is a side view of an alternative embodiment of the device of FIG. 1, shown with a retractable blade which can be used in accordance with a preferred embodiment of the invention. FIG. 3a is an expanded view of the blade from FIG. 3.

FIG. 4 is a side view of another embodiment of the surgical guide device of the present invention shown with a blade mounted on a guide arm thereof and without a blade disposed between the guide arms.

FIG. 5 is yet another exemplary embodiment of the surgical guide device of the present invention.

FIG. 6 shows a preferred embodiment with the interior edges of the device shaped to snuggly fit together.

FIG. 7 is an aerial view of a person's palm which exposes the carpal ligament in close proximity to the median nerve and further shows an application of the surgical guide device to release the transverse carpal ligament.

FIG. 8 is an aerial view of the hand post operation, shown with an ice pack inserted into a surgery incision.

FIG. 9 is a side view of a surgical guide device constructed in accordance with yet a further preferred embodiment of the present invention.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

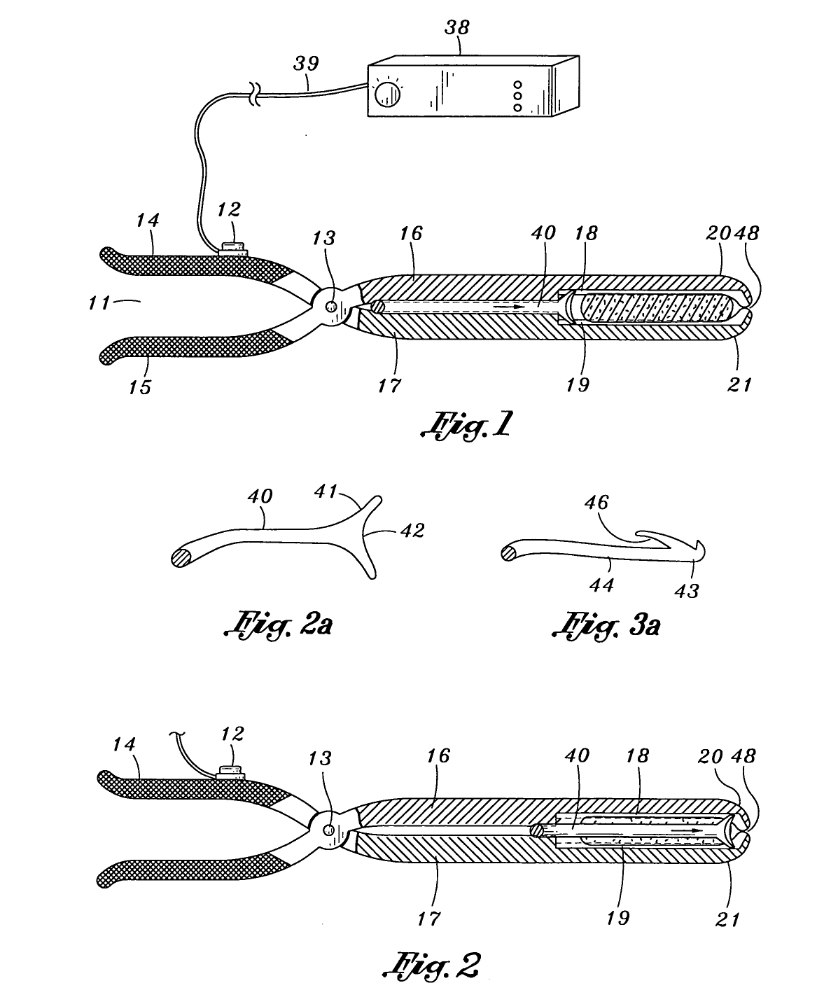

Referring now to the figures, and initially to FIG. 1, there is shown a device for releasing a ligament that comprises an electrical source 38 that is electrically connected to a surgical guide device 10. The surgical guide device 10 has an activation means, such as a button 12, which permits an electrical current to flow from the electrical source to the surgical guide device. The surgical guide device 10 comprises a first guide arm 16 that has a distal end 20 and a proximal end 14. The first guide arm also has an interior edge 18 that runs along a longitudinal axis of the guide arm. The first guide arm 16 is coupled to a second guide arm 17 that is of substantially similar shape as the first guide arm. The second guide arm 17 also has a distal end 21, proximal end 15, and an interior edge 19. In a preferred embodiment, the second arm is coupled to the first arm by a common pivot point 13. The proximal ends of the guide arms 16, 17 extend past the pivot point 13 to form a handle 11 for a surgeon to grasp during surgery. The handle may be compressed to move the distal ends 20, 21 of the guide arms toward and away form each other, for relative movement between an open, closed, and intermediate position. In a preferred embodiment, the distal ends of the guide arms form a tip 48 to prevent a blade 40 from extending beyond the device 10.

The guide arms are coupled such that the interior edges 18, 19 form facing edges. At least one of the guide arms supports a nerve stimulation electrode that is electrically connected to the activation means 12 on the device 10. Electrical current runs through the electrode into the distal end of the guide arm. As explained in more detail below, a ligament will be disposed between the interior edges of the device. If a nerve is inadvertently enclosed within the interior edges, and comes into contact with the nerve stimulation electrode, the nerve will send an impulse to a muscle causing the muscle to twitch. In a preferred embodiment, the interior edge 18 may be lined with an adhering conductive material, such as metal, to transmit electricity along the interior edge of the first arm 16. In yet another preferred embodiment, both interior edges 18, 19 are lined with a conductive material and act as nerve stimulation electrodes.

In the preferred embodiment shown in FIG. 1, a blade 40 is disposed between the interior edges 18, 19 of the device arms near the handle 11 or the pivot point 13 of the device. Referring to FIG. 2a, the blade has an arcuate surface 42 along its distal end 41 that forms a sharp edge. Referring now to FIG. 2, when a ligament is disposed between the interior edges 18, 19 of the surgical guide device, the blade 40 may be projected toward the distal ends 20, 21 of the surgical guide arms and away from the guide handle 11. When the blade 40 is projected, its sharp edge slices through the ligament. Once the ligament is cut, or released, the blade may be retracted back toward the pivot point 13 to a resting position.

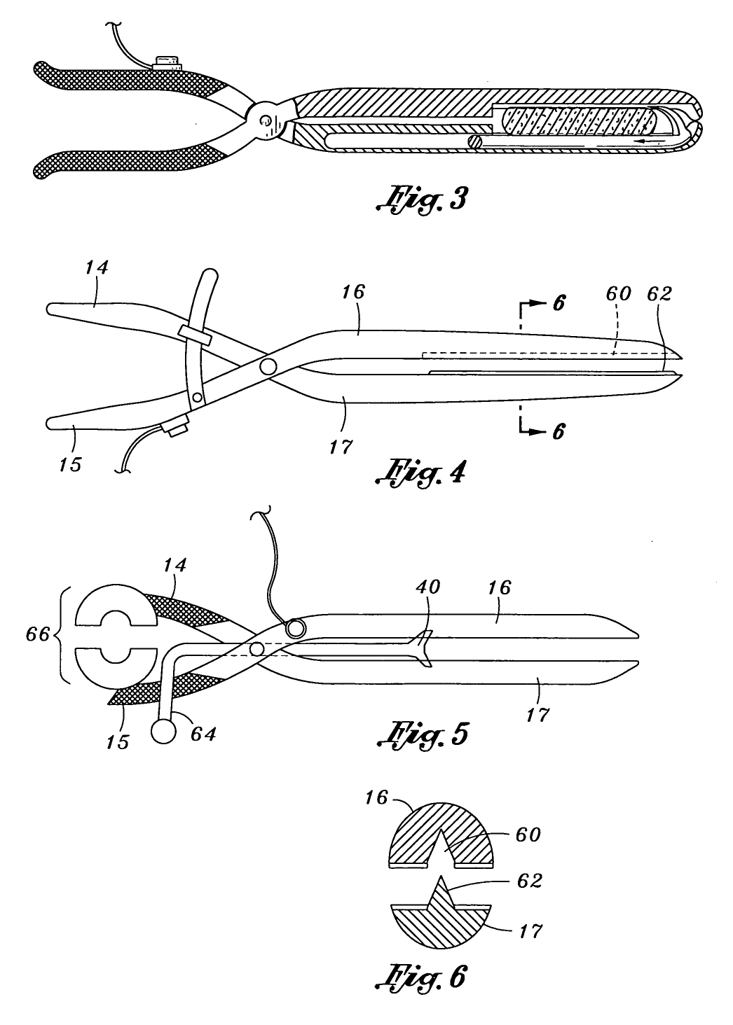

FIGS. 3 and 3a depict the surgical guide device with an alternative blade 44. The blade 44 forms a hook at its distal end 43. The inner surface of the hook forms a sharp edge 46 capable of cutting through ligament and tendon. Referring now to FIG. 3, when a ligament is disposed between the interior edges 18, 19, the blade may be projected toward the distal ends 20, 21 of the surgical guide arms. Pulling the blade back toward the guide handle 11 exposes the ligament to the blade's sharp edge 46 and cuts the ligament.

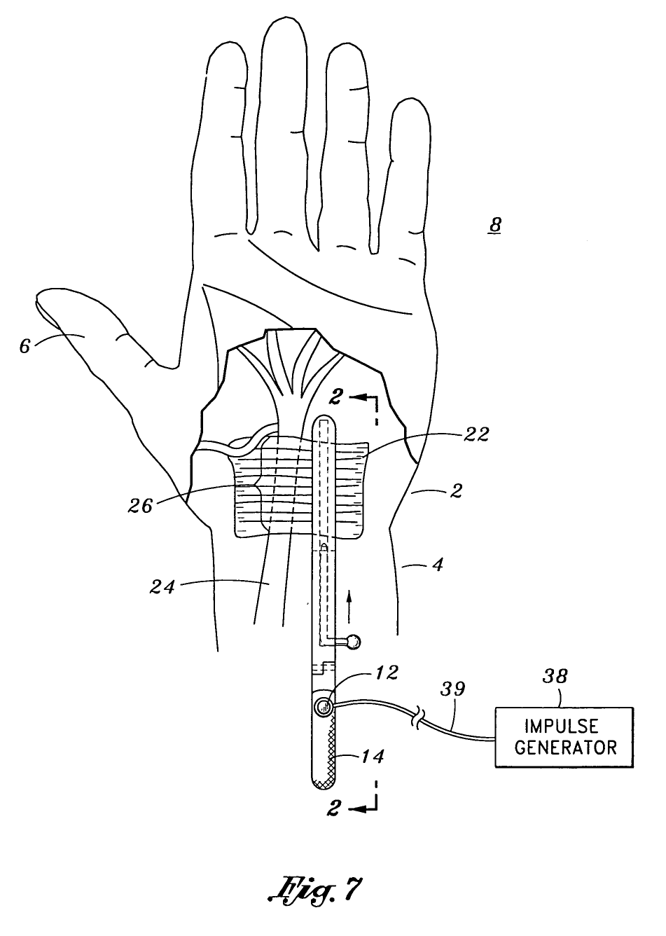

FIGS. 4, 5 and 9 show alternative embodiments of the surgical guide device of the present invention. Specifically, FIG. 4 depicts a scissor-type embodiment wherein the cutting action is provided by an elongate blade section 62 formed on guide member 17 as operatively received within recess 60 on guide arm 16. As depicted in FIG. 6, cutting element 62 is received within recess 60 and is thus operative to cut a section of ligament disposed therebetween. FIG. 5 depicts an embodiment similar to FIG. 1 with the blade 40 being operatively advanced via handle 64. Moreover, such embodiment depicts that proximal ends 14, 15 may be secured into position via clamp mechanism 66. FIG. 9 depicts an embodiment comprising the operative combination of a handle mechanism 68 in operative combination with an elongate cutting knife with handle. Such elements 68, 70 are operatively interconnected with one another to make elongate cutting motions. To that end, handle mechanism is provided with an elongate grooved limb 72 that is operative to serve as a guide for introduction of the cutter 70. The grooved limb 72 is further provided with a blunt end with stop 74 to prevent inadvertent cutting and/or tissue damage as the instrument is advanced. A blunt protectant is also formed on the leading edge of cutting knife handle 70 that cooperates with blunt end 74 to prevent inadvertent cutting and/or tissue damage.

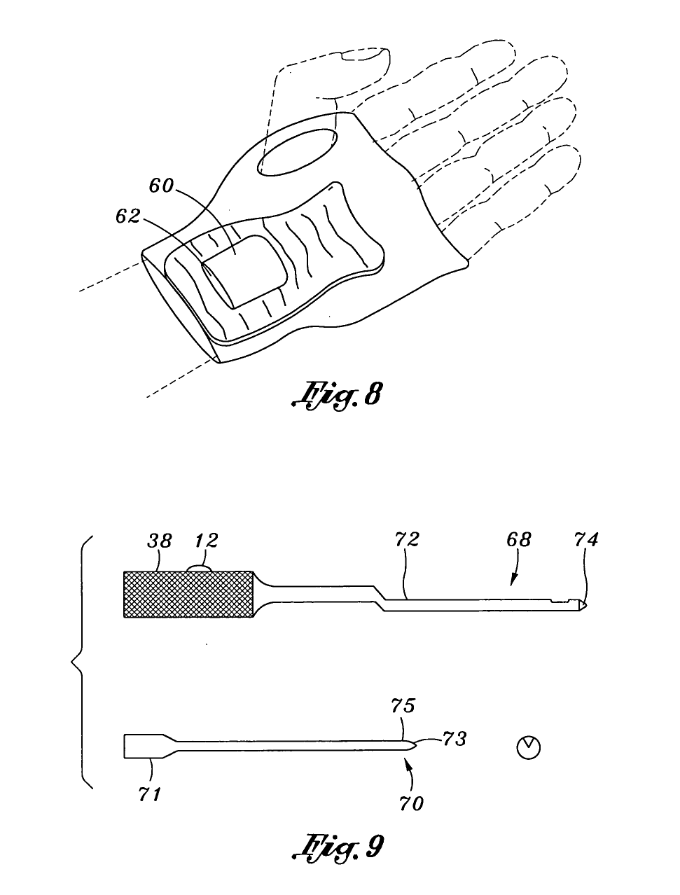

FIG. 7 shows an application of the surgical guide device to release a ligament. Specifically, FIG. 4 illustrates an application of the surgical guide device 10 to release the transverse carpal ligament 22. FIG. 4 is an aerial view of hand 8, palm 2, and device 10, and highlights the close proximity with which the transverse carpal ligament 22 is located in relation to the median nerve 24. The carpal tunnel 26 is formed from ligaments and bones including the transverse carpal ligament 22. The median nerve 24, as well as other tendons which are not shown, pass through the carpal tunnel 26. As explained in the background section, carpal tunnel syndrome develops when the median nerve 24 becomes compressed within the tunnel. The objective of open release surgery is to cut the carpal ligament to reduce pressure on the median nerve. In open release surgery, a doctor typically makes a one and one half to two and one half inch incision extending from the base of the palm 2 away from the wrist 4. Using current techniques, the doctor cuts the exposed ligament, but the doctor cannot be certain that a nerve will not inadvertently be severed. The present invention alleviates this grave danger by introducing a surgical guide device to stimulate nerves before cutting through a ligament. By using the present invention, a doctor will know with certainty whether she will not slice through a nerve.

Referring now to FIGS. 7 and 1, the surgical guide device of the current invention is inserted into the incision. The distal ends of the first and second guide arms (FIG. 1, elements 16, 17) should be slightly open. The surgeon positions the guide arms around the ligament 22 so that the ligament is enclosed between the interior edges 18, 19 of the first and second guide arms. The surgeon introduces an electrical signal to the device by depressing the activation button 12 fastened on the device. The electrical current transmitted to the device will range from 0.5 to 2.0 mV. The electrical current is transmitted to at least one of the nerve stimulation electrodes located on the distal ends 20, 21 of the guide device arms 16, 17. In a preferred embodiment, the nerve stimulation electrode comprises a metal, or other conductive material, integrally formed to the interior edges of at least one of the first or second guide arms. If the nerve stimulation electrode comes into contact with the median nerve 24, or any other adjacent nerve, the nerve will transmit an electrical impulse to a muscle, causing that muscle to move. In the case of surgery on the transverse carpal ligament, the thumb 6 may twitch in response to electrical stimulation of the median nerve. The surgeon can then reposition the surgical guide device arms 16, 17 around the ligament 22 such that the median nerve 24 is not entrapped between interior edges 18, 19 of the device and is not in danger of being severed.

Once the proper alignment has been established, a blade 40 is then projected from a point near the common pivot point 13 toward the distal ends 20, 21 of the blade guide arms to cut the ligament. A blade of an alternate shape, such as one discussed above and shown in FIG. 3a, or a scissors type blade, can also be used in the present invention. It should further be expressly recognized that other types of cutting means can be utilized in the practice of the present invention. Specifically, it is contemplated that the ligament can be cut using electrocautery, either standard or bipolar, radiofrequency, and other types of blades, scissors, and the like.

FIG. 8 shows a hand post operation on the transverse carpal ligament. An ice pack 60 is inserted into the incision to aid the ligament in healing and its perforated base 62 provides an opening through which the median nerve passes.

Additional modifications and improvements of the present invention may also be apparent to those of ordinary skill in the art. For example, the devices of the present invention may be operative to simply remove a strip of ligament instead of just incising the ligament. In this regard, any cutting technique that is operative to reduce pressure exerted by the transverse carpal ligament on the median nerve should be deemed to fall within the scope of the present invention. Thus, the particular combination of parts and steps described and illustrated herein is intended to represent only certain embodiments of the present invention, and is not intended to serve as limitations of alternative devices and methods within the spirit and scope of the invention. Along these lines, it is contemplated that the device may be used to release ligaments, tendons, or connective tissue that are positioned adjacent to, or dangerously close to, a nerve. The present invention may be particularly suited for deep plane face lifts where the surgery produces high risk of damage to facial nerves.

What is claimed is:

1. A device for cutting a ligament, the ligament being disposed adjacent a nerve, the device comprising:
an elongate first blade guide arm defining a longitudinal axis, the first blade guide arm having a distal end;
an elongate second blade guide arm defining a longitudinal axis, the second blade guide arm having a distal end and being coupled to the first blade guide arm;
a nerve stimulation electrode supported by the distal end of at least one of the first or second blade guide arms and being energizeable to stimulate a nerve upon contact with the nerve; and
a blade engaged to at least one of the blade guide arms, the blade formed to move parallel to the longitudinal axis adjacent to the distal end of the blade guide arm for cutting the ligament.

2. The device of claim 1 wherein the first blade guide arm is coupled to the second blade guide arm by a common pivot point for relative movement of the distal ends toward and away from each other.

3. The device of claim 2 wherein the first and second blade guide arms have proximal ends, the proximal ends extending past the common pivot point to cooperatively form a handle.

4. The device of claim 3 wherein the handle supports a conductor electrically connected to the nerve stimulation electrode to transmit an electrical signal.

5. The device of claim 1 wherein the first and second blade guide arms have facing interior edges for contacting a nerve prior to cutting the ligament.

6. The device of claim 5 wherein the nerve stimulation electrode comprises a conductive metal integrally formed to at least one of the interior edges of the blade guide arms.

7. The device of claim 1 wherein the blade is retractable.

8. The device of claim 7 wherein the retractable blade has a proximal end and distal end, the distal end having an arcuate surface, the arcuate surface having a sharp edge for cutting a ligament when the blade is projected toward the distal ends of the first and second blade guide arms.

9. The device of claim 7 wherein the retractable blade has a proximal end and distal end, the distal end forming a hook wherein the hook comprises a sharp edge for cutting a ligament when the blade is pulled away from distal ends of the first and second blade guide arms.

10. The device of claim 1 wherein the distal ends of the first and second blade guide arms form a tip for halting the blade.

11. A method for cutting a ligament, the ligament being disposed adjacent a nerve, the method comprising the steps of:
a. connecting an electrical source to a surgical guide device, the surgical guide device comprising:
an elongate first blade guide arm defining a longitudinal axis, the first blade guide arm having a distal end;
an elongate second blade guide arm defining a longitudinal axis, the second blade guide arm having a distal end and being coupled to the first blade guide arm;
a nerve stimulation electrode supported by the distal end of at least one of the first or second blade guide arms and being energizeable to stimulate a nerve upon contact with the nerve; and
a blade engaged to at least one of the blade guide arms, the blade formed to move parallel to the longitudinal axis adjacent to the distal end of the blade guide arm for cutting the ligament;
b. making an incision adjacent to a proximal and distal edge of a ligament;
c. positioning a portion of the surgical guide device into the incision such that the ligament in step (b) is disposed between the first and second blade guide arms of the surgical guide device;
d. transmitting an electrical signal to the nerve stimulation electrode of the surgical guide device;
e. monitoring a patient for a motor response to verify that the surgical guide device in step (d) is not positioned such that a nerve is disposed between the first and second blade guide arms; and
f. introducing the blade of the surgical guide device in step (a) to release the ligament.

11. The method of claim 10 wherein the ligament in step (b) is the carpal tunnel ligament.

12. The method of claim 10 wherein step (e) further comprises repositioning the surgical guide device so that the patient does not exhibit a motor response.

13. The method of claim 10 wherein in step (f), the ligament is released by projecting the blade toward the distal ends of the first and second blade guide arms.

14. The method of claim 10 wherein in step (f), the ligament is released by retracting the blade away from the distal ends of the first and second blade guide arms.

15. The method of claim 10 wherein in step (f), the distal ends of the first and second blade guide arms form a tip for halting the blade.

16. The method of claim 10 wherein in step (d), the electrical impulse transmitted is between 0.5 and 2.0 mV.
